(19) United States
(12) Patent Application Publication
(10) Pub. No.: US 2015/0335695 A1
(43) Pub. Date: Nov. 26, 2015

(54) Therapeutic Nutritional Supplement to Alleviate Symptoms of COPD and Delay the Progression Thereof

(71) Applicant: Tu Song-Anh Nguyen, Westminster, CA (US)

(72) Inventor: Tu Song-Anh Nguyen, Westminster, CA (US)

(21) Appl. No.: 14/718,043

(22) Filed: May 20, 2015

Related U.S. Application Data
(60) Provisional application No. 62/002,905, filed on May 25, 2014.

(51) Int. Cl. A61K 36/481; A23L 2/52; A61K 36/074; A23L 1/29; A61K 31/12; A61K 31/353

(52) U.S. Cl. CPC A61K 36/481; A61K 31/12; A61K 31/353; A61K 36/074; A23L 1/293; A23L 2/52; A23V 2002/00

(57) ABSTRACT

A therapeutic nutritional supplement includes a predetermined amount of Astragalus to alleviate the symptoms of COPD with its antioxidant and anti-inflammatory effect and its inhibitory action on the goblet cells to attenuate coughing, and to delay the progression of COPD with the activation for stem cells to promote the healing efficacy for injured lung tissue; and a predetermined amount of Curcumin to alleviate the symptoms of COPD with its synergistic antioxidant and anti-inflammatory effect, and to delay the progression of COPD with the inhibitory action on the production of the enzyme elastase to prevent the damage of alveoli and to reverse the epigenetic modification caused by chronically exposing to tobacco smoke, an air-polluted, and an chemical fume environment, wherein the nutritional supplement can be utilized in beverages, foods, supplements, and drugs.

### CROSS REFERENCE OF RELATED APPLICATION

This is a non-provisional application that claims priority to U.S. provisional application, application No. 62/002,905, filed May 25, 2014.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to a nutritional supplement, and more particularly to a therapeutic nutritional supplement containing a predetermined amount of Astragalus and Curcumin to alleviate the symptoms of chronic obstructive pulmonary diseases (COPD) and delay the progress of this disease.

2. Description of Related Arts

Chronic obstructive pulmonary disease (COPD) is also known as chronic obstructive lung disease (COLD) or chronic obstructive airway disease (COAD), wherein it is characterized as the following three symptoms: the shortness of breath, cough, and the production of mucus. COPD is a disease that results in poor quality of life due to chronic dyspnea, a condition of shortness of breath like a fish out of the water. Just I imagine how much you have suffered from coughing and mucus production from a flu for a week. People with COPD suffer with dyspnea every day for the rest of their life. Unlike asthma, these symptoms do not improve significantly with the administration of current medications. Furthermore, COPD only progresses worse over time.

According to the report from the World Health Organization, there were 329 million people worldwide (nearly 5% of total population in the world) suffering from COPD in 2011, wherein COPD is ranked as the 4^{th} disease for commonly causing of death and there are 2.1 trillion dollars recorded as economic loss in 2010. The projected number of people having COPD will be higher and higher in the future because of the serious air pollution especially in the developing countries in Asia. As a result, the number of deaths being caused by COPD is also projected to be increased due to the increase of an aging population and smoking rates worldwide.

Smoking, air pollution, and exposing in an air-polluted occupational environment are three main factors for causing COPD. The highest risk factor for developing COPD is smoking since about 80% of people having COPD are current smokers or previous smokers. According to the statistic, about 20% of the smokers will eventually have COPD, and of those who are lifelong smokers about half will get this disease. Obviously, the smokers have a high possibility to have COPD. Unfortunately, the innocent bystanders who don't smoke, such as the secondhand smokers, also have a high possibility to have COPD. According to the above described academic statistic, up to 20% of the patients having COPD are secondhand smokers. In other words, one of five patients suffering from COPD is because that he or she is unlucky enough to be around with smokers. Another significant factor for developing COPD is that people chronically expose around the air pollution. The number of the people having COPD which is caused by being chronically exposed around the air pollution is projected to be much higher in the next 30 years, due to that the air quality in many developing countries around the world is getting worse. Air pollution is one of the most common factors for causing COPD in many developing countries. Finally, exposing in the air-polluted occupational environment is another common factor for causing COPD. Recently, many reports have shown the increase of the risk for having COPD by staying around harmful fumes in nail salons, which include silica, methyl methacrylate (acetone, toluene, etc . . . ), and formaldehyde. The silica dust has been found to significantly increase the risk of having COPD. According to a research done by University Hospital of Mont-Godinne in Belgium in May 1999, even if a person is exposed in a low level of gas toluene environment in a brief 6 hours, the conductance of airway will be significantly decrease with many cellular changes. Majority of nail salon workers suffer from symptoms of COPD, such as headache, eyes and noses irritation, and breathing problems due to the prolonged exposure in the harmful fumes. According to American Journal of Public Health in 2011, toxic fumes, methyl methacrylate, are found in Vietnamese nail salons, which is a compound that have been banned from nail products. According to another research done by Health Department of Utah, USA, “methyl methacrylate was detected in 58% of the establishments despite having been banned for using in nail products by the state of Utah”.

The pathophysiology of COPD consists of chronic bronchitis and emphysema. The key problem in emphysema is the destruction of elastin (the elastic connective tissues such as in lung, bladder, blood vessels, skin, etc . . . ) by the enzyme elastase, which is hyperactive in response to the chronic inflammation. In another words, long term exposure to smoking, pollutants, or toxic fume cause the lining of aveoli, the tiny air sacs where oxygen and carbon dioxide exchange, to be irritable and inflamed, then eventually being destroyed. On the other hand, the main problems in chronic bronchitis are the destruction and narrowing of the main airways (brochioles and bronchi), the increase of goblet cells' activities resulting constant mucus production, and cough. The analogy for the pathophysiology of COPD is like when you constantly rub irritant materials on the skin of your body. Some parts of your skin become ulcerated with the irreversible destruction of skin like that of emphysema. Some other parts of your skin turn into scars like those of chronic bronchitis, of which the main airways are thickened and narrowed with hyperactive mucus production. It may take 20-30 years for COPD to develop, but once it appears, it is irreversible and progressively worse overtime.

Despites all the modern medical technologies nowadays, there are no cure for COPD. Unfortunately, patients with COPD are forever suffering from chronic coughing and the shortness of breathing in his or her whole life, such like a fish being taken out of its tank of water until the day they die. Worse, unlike other diseases, such as cancer that may kill them soon, patients with COPD may live for a long time but endlessly suffer from symptoms thereof every second of their life. Since there are no method and no effective treatment to completely cure for COPD, the best treatment is the prevention, which means of quitting smoking, staying away from pollution which can induce COPD, and finding another job where you don't need to be exposed around the air pollutants. It is difficult for people who can't quit smoking, can't get away from the smokers, can't move away from air-polluted city, and can't quit their job. Therefore, the present invention provides a therapeutic nutritional supplement, which is capable to alleviate the symptoms of chronic obstructive pulmonary disease (COPD) and delay the progress of this disease

### SUMMARY OF THE PRESENT INVENTION

A main object of the present invention is to provide a therapeutic nutritional supplement for alleviating the symptoms of chronic obstructive pulmonary disease (COPD) and delaying the progress of this disease, wherein the therapeutic nutritional supplement comprises Astragalus and Curcumin.

Another object of the present invention is to provide a therapeutic nutritional supplement for alleviating the symptoms of chronic obstructive pulmonary disease (COPD), wherein Astragalus is able to decrease the activities of goblet cells and the production of the mucus producing cells, which are responsible for the constant production of phlegm and mucus for chronic bronchitis.

Another object of the present invention is to provide a therapeutic nutritional supplement for delaying the progress of COPD, wherein Astragalus is able to markedly inhibit the remodeling of the airway, including the fibrosis of sub-epithelial, the hypertrophy of smooth muscle, and the hyperplasia of goblet cells via the activations of stem cells.

Another object of the present invention is to provide a therapeutic nutritional supplement for delaying the progress of COPD, wherein the stem cells are activated by Astragalus to generate and repair injured lung tissue via the following three mechanisms: the rejuvenation of the stem cells by enhancing the amount of the enzyme telomerase, the proliferation of stem cells, and the differentiation of stem cells.

Another object of the present invention is to provide a therapeutic nutritional supplement for alleviating the symptoms of COPD, wherein Curcumin is a potent antioxidant that helps to decrease airway injury and decrease the production of mucus therein.

Another object of the present invention is to provide a therapeutic nutritional supplement for alleviating the symptoms of COPD, wherein Curcumin is a powerful elastase inhibitor, which prevents the elastin from being destroyed by the enzyme elastase, wherein the elastin is an elastic connective tissue that is responsible for the respiration in the lung.

Another object of the present invention is to provide a therapeutic nutritional supplement for delaying the progression of COPD, wherein Curcumin is a regulator for epigenetic events so as to specifically reverse the epigenetic modification caused by chronically exposing in a smoking, pollution, and toxic fume environment according to a study published by University of Rochester Medical Center, USA.

Another object of the present invention is to provide a therapeutic nutritional supplement, which can be used in therapeutic drugs, medications, herbal supplements, beverages, and foods for human.

Another object of the present invention is to provide a therapeutic nutritional supplement, wherein the two components, Astragalus and Curcumin, are easy to obtain, cheap in prices, and simply to incorporate with drugs, foods, and beverages so as to provide a better nutritional supplement for alleviating the symptoms of COPD and delaying the progress of COPD.

Additional advantages and features of the invention will become apparent from the description which follows, and may be realized by means of the instrumentalities and combinations particular point out in the appended claims.

In order to achieve the above described objects, the present invention provides a therapeutic nutritional supplement to alleviate symptoms of COPD and delay the progression thereof, comprising:

a predetermined amount of Astragalus having a daily dosage range from 100 mg to 2000 mg; and

a predetermined amount of Curcumin having a daily dosage range from 10 mg to 300 mg.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a block diagram according to a preferred embodiment of the present invention, illustrating the composition of the present invention.

FIG. 2 is a block diagram according to the above preferred embodiment of the present invention, illustrating the therapeutic effect of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

The pathophysiologies of chronic obstructive pulmonary disease (COPD) are chronic bronchitis and emphysema. The factor for causing emphysema is the destruction of the elastin, which are elastic connective tissues found in our lung, by the enzyme elastase in response to the chronic inflammation. And, the factor for causing chronic bronchitis is the destruction and narrowing of the main airways and the increase of activities of goblet cells, mucus producing cells, which causes chronically coughing. The main objective of the present invention is to provide a therapeutic nutritional supplement for (1) alleviating the symptoms of coughing by inhibiting the production of the mucus producing goblet cells and using a potent antioxidant to attenuate the ongoing inflammatory process in our lung; (2) and delaying the progress of COPD by the following three mechanisms: using elastase-inhibiting agents to hinder the incessant destruction of elastin (which causes emphysema), using a regulator for epigenome to reverse the epigenetic modification, caused by chronically exposing to a smoking, pollution, and toxic fume environment, and using natural agents to activate and rejuvenate stem cells, which can repair and replace the injured lung tissues.

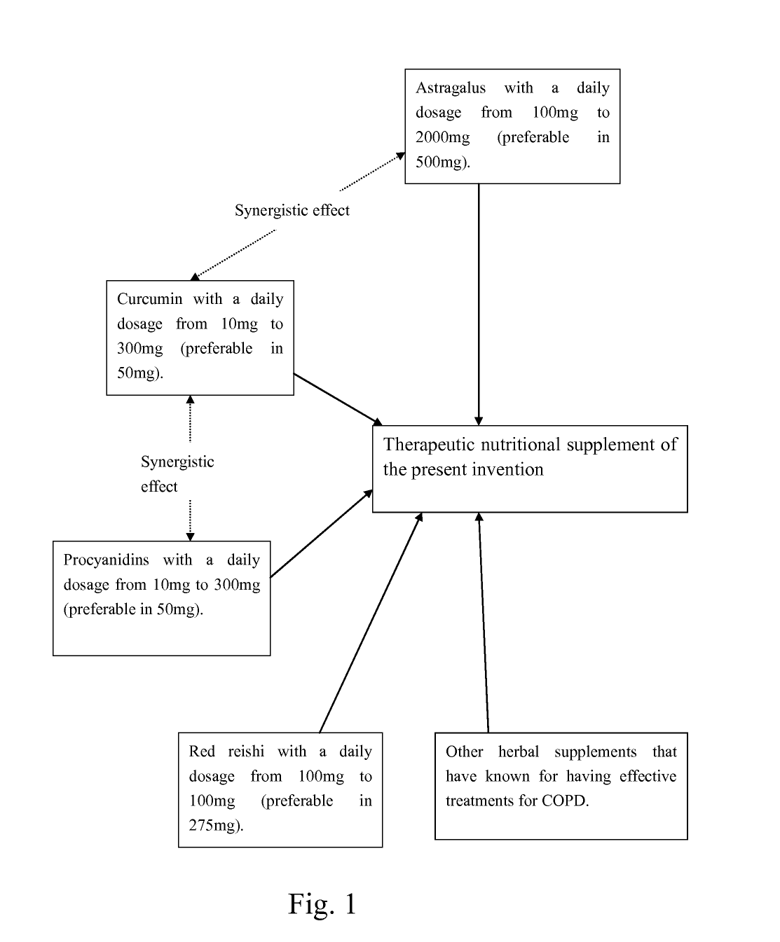

Referring to FIG. 1 of the drawings, a therapeutic nutritional supplement according to a preferred embodiment of the present invention is illustrated, wherein therapeutic nutritional supplement comprises two components, which are Astragalus and Curcumin, wherein a daily dosage range for Astragalus is from 100 mg to 2000 mg, and preferably, the daily dosage thereof is about 500 mg. And, a daily dosage range for Curcumin is from 10 mg to 300 mg, and preferably, the daily dosage thereof is about 50 mg.

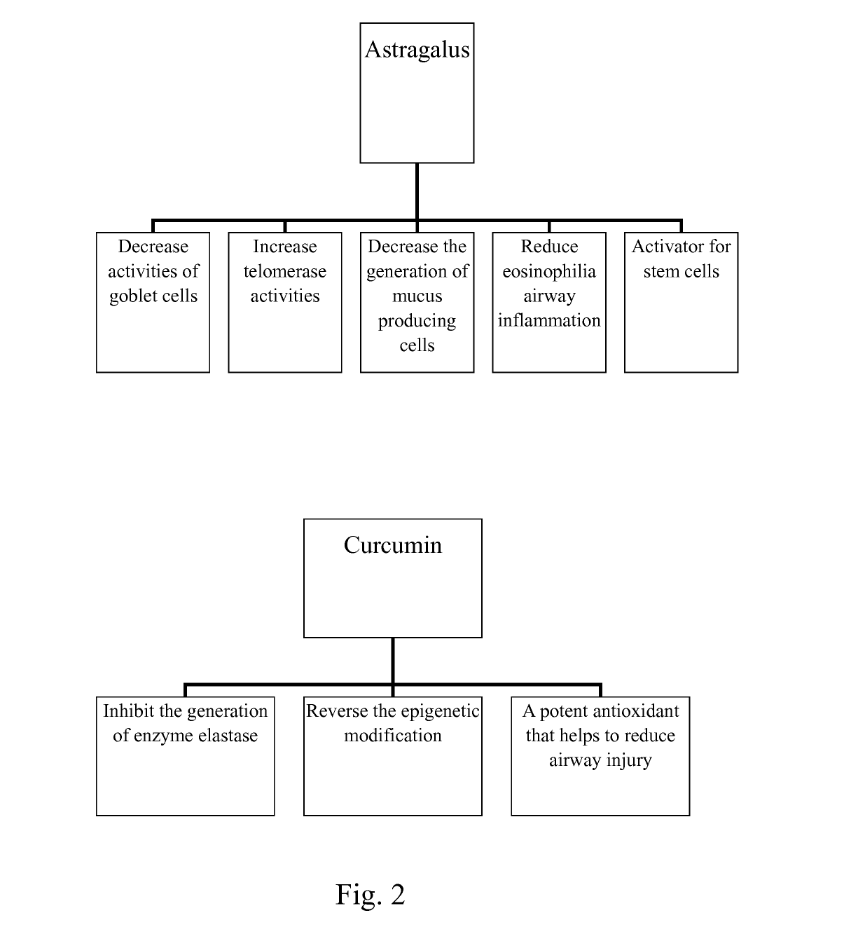

Referring to FIG. 2 of the drawings, Astragalus (Astragalus membranaceus) was shown to decrease the activities of goblet cells, the mucus producing cells that are responsible for constant production of phlegm and mucus of chronic bronchitis, resulting significant improvement of coughing symptoms. According to a research published on July 2008, Astragaloside IV, a triterpene glycoside extracted from Astragalus membranaceus, is responsible for reduction of eosinophilic airway inflammation. In other words, Astragalus helps to decrease the allergic inflammatory reaction that stimulates the increase in activity of the mucus producing goblet cells. In a study that was designed to explore the role of Astragalus in the pathogenesis of bronchopulmonary dysplasia in preterm babies, Astragalus was demonstrated to retard the inflammatory response, which may eventually cause lung tissue destruction.

Curcumin, a chemical found in the popular South Asian spice turmeric, which is a member of the ginger family, is also a potent antioxidant that helps to reduce airway injury in chronic bronchitis, according to a study by VA Hospital in USA in September 2012. Furthermore, Curcumin is one of the natural agents that inhibit the enzyme elastase¹⁶. In responding to the chronic inflammatory process, the enzyme elastase is released by the neutrophils. Elastase breaks down elastin, the elastic connective tissues that are abundant in the aveoli (the tiny sacs that allow the exchange of oxygen and carbon dioxide inside our lungs.) Emphysema develops when elastin is broken down. Therefore, the more elastin is destroyed, the worse symptoms of the shortness of breath are, and the poorer quality of life of the patients will be.

As shown in FIG. 1, the therapeutic nutritional supplement further comprises other elastase-inhibiting agents, which can be used together with Curcumin for the advantageous synergistic effect. One of the elastase-inhibiting agents is Procyanidins (also known as Proanthocyanidins), wherein a daily dosages range of Procyanidins is from 10 mg to 300 mg, and preferably, the daily dosage thereof is about 50 mg. Procyanidins is a flavanols, which is found in a high concentration in the bark of pine (for example, marine time pine), cinnamon, and grape seeds. Besides the antioxidant activity of the Procyanidins, it is found to be a very potent elastase inhibitor that helps to delay the destruction of the lung tissue in emphysema, causing by tobacco smoke, heavy pollution, and chemical fume. Procyanidins also plays a role to stabilize the collagen and to maintain the amount of elastin in our lungs. Furthermore, it is also found to protect the lung from being damaged in heavy smokers. Procyanidins may be used with Curcumin to generate the synergistic effect.

The therapeutic nutritional supplement further comprises red reishi, which is a fungal species of the genus Ganoderma, wherein the red reishi is used to be a medicinal mushroom in traditional Chinese herbal medicine. According to the recent study, red reishi is proven to enhance our immune system and have high efficacy for alleviating the symptoms of COPD. A daily dosage of red reishi is from 100 mg to 1000 mg, and preferably, the daily dosage of the red reishi is 275 mg.

It is worth mentioning that another mechanism to delay the progression of COPD is utilizing a regulator of epigenome to reverse the epigenetic modification caused by chronic exposure to smoking, pollution, and toxic fume. Epigenetic modification is a heritable change in genes activity that is not caused by changes in DNA sequences. Human DNA is about 6 feet long, but it can be tightly coiled to fit inside a microscopic cell. It is capable to do so because it wraps around proteins known as histones and continues to wrap further around itself again and again into a microscopic chromosome. A very tightly wrapped segment of DNA can't be accessed; therefore, the genes within this segment of DNA can't be read. When a certain protein or enzyme within this segment of DNA needs to be made for the cell to use, the tightly wrapped DNA unwinds itself to allow room for it to be read and transcribed. Epigenetic modification is a process, of which the histones are modified via acetylation or methylation (an addition of methyl group CH₃—); consequently, the DNA segment wrapping around the modified histones can't be unwound, and the cell can't make that protein or enzyme.

Many internal and external factors such as toxins, pollutants, chemicals, fumes, radiation, and diets and so on can modify the protein histones. Let's use a set of twin for illustration. A set of twin right after birth can look very alike because they both share the identical set of DNA. When the twins are growing up, each twin has different habit. For example, they eat, drink, and expose themselves in different environment. Overtime, the histones in each twin are modified differently. That is to say, the more remarkable difference among the twins is that the same gene from the twins is expressed (turned on) in one twin but it turned off in the other twin. Noticeably, if the twins are separated after birth and being raised by two different families, they will look very dissimilar because each set of genes from each twin are expressed even more differently.

While the DNA is unchanged, modification of the histones can significantly alter the expression of the genes in different individuals. For example, according to a research published by University of Southwestern Medical Center in Dallas, USA in November 2013, tobacco smoking was shown to modify the histones group by methylation of the gene P16, which is known as cyclin-dependent kinase inhibitor 2A, multiple tumor suppressor 1, or a tumor suppressor protein encoded by the CDKN2A gene. In another words, CDKN2A gene after being read and translated would produce a tumor suppressor protein P16, which does exactly as what it says: suppressing cancer. Tobacco smoke turns off this gene by methylating the histones, of which the segment of this P16 gene is wrapping around; thus, it prevents this tumor suppressor protein to be produced. Consequently, there is much higher risk of getting lung cancer because there is no tumor suppressor protein P16 to suppress it from happening. Unfortunately, when tobacco smoke, pollution, or toxic fumes turn off the tumor-suppressing gene, it increases the risk of lung cancer not only in your lifetime but also in your unborn children, grandchildren, and great grandchildren. It does so because the P16 gene on your gonadal line of cells (sperms and eggs) are also affected and being modified. In other words, whatever you do affects not only you yourself but also many of your generation in epigenetic modification process. Besides the higher risk of cancer development, the oxidative stress from chronic exposure to smoking, pollution, or fume is shown to modify the histone acetylation, which activates genes that cause COPD by increasing elastase activity in emphysema and airway destruction in chronic bronchitis, according to a study “Current concepts on oxidative/carbonyl stress, inflammation and epigenetics in pathogenesis of chronic obstructive pulmonary disease” published on July 2011.

According to the preferred embodiment of the present invention, Curcumin is an epigenetic modulator that can turn on or turn off the P16 gene. For example, Curcumin is shown by many researches to act as a DNA hypomethylating agent or an inhibitor of DNA methyltransferase, which is an enzyme to methylate the histone for turning off the P16 gene. Therefore, if a person ingests Curcumin daily, the tumor suppressing gene P16 will turn on again, so the risk of having lung cancer for that person will decrease, and this result is not only in his or her lifespan but also in the life of his or her unborn children and grandchildren. Moreover, Curcumin is also shown to affect histone deacetylase that is related to many cellular inflammatory responses causing from the progression of COPD. That is to say, Curcumin can reverse the epigenetic modification caused by chronically exposing in the smoking, air-polluted, and toxic fume environment according to a study published by University of Rochester Medical Center, USA.

In summary, Curcumin, a chemical found in turmeric (Curcuma longa), possesses three miraculous properties. First of all, it is a potent antioxidant that helps to reduce airway injury in chronic bronchitis. Secondly, it is an elastase inhibitor that prevents the destruction of aveoli in emphysema. In fact, it is so effective that according to one study, eating rich curcumin-curry diet was found to preserve pulmonary function in heavy smokers. And finally, Curcumin can reverse the epigenetic modification caused by the chronic exposure to tobacco smoking, air pollution, and chemical fume.

It is worth mentioning that while the progression of COPD is delayed by Curcumin, a regulator of epigenome, Astragalus perform a different mechanism at the same time. Astragalus is an activator for stem cells. Millions of cells in our body die every day; thus, we need to make new cells to replace the death ones constantly. The special cells that are responsible for regenerating the new cells are known as stem cells. For example, the entire lining of our intestinal tract is completely replaced for every 4 to 5 days while the stem cells reside in the crypts of Lieberkuhn. Stem cells have three unique characteristics that make them very special. First of all, they can multiply to make more of the same stem cells almost forever while the mature regular cells can only divide and multiply for only up to 50 times. Secondly, they can differentiate to become many other types of cells. For example, undifferentiated hematopoietic stem cells can differentiate to become either white blood cells or red blood cells. Finally, they can repair injuries by regeneration. For example, when the skin of a fetus is cut open, the wound repairs flawlessly without any signs of scar because the skin is repaired by a regenerating mechanism. When we are infants, we have a lot of stem cells available. Unfortunately, as we are growing older, the numbers of stem cells available in our body decrease. They are not available to repair damaged tissues effectively. That is the reason why an infant's skin can heal very well without scar via regeneration, but an adult's skin can still heal but with a big thick scar. Due to aging and barely having enough stem cells to replace the death ones, an elder's skin can't heal well at all. The wound may stay open and bleeding for a long time.

Stem cells can stay alive and keep dividing and dividing almost forever because they have telomerase, an enzyme that makes telomeres. Telomere is a region including repetitive nonsense of nucleotide sequences at the ends of each chromatid (DNA), wherein telomere can protect the chromosome from being deteriorated. For example, a chromosome is like a shoelace, and the telomeres are the aglets, which are the metal or plastic tags at both ends of each shoelace. The aglet makes the shoelace to lace easier, and also protects the shoelace from unraveling. Therefore, the relationship between telomeres and the chromosome are like that of the aglets and the shoelace. When a cell divides, it duplicates its contents of chromosomal DNA, and the ends of the chromosomes get chopped off and become a little bit shorter. A mature normal cell also has telomeres; however, when the mature normal cell divides, its telomeres get shorter and shorter each time. After about 40 to 50 divisions of the mature cell, the mature cell will die when it runs out of its telomeres. Unlike the mature cell as described above, a stem cell can keep dividing and dividing above 50 times because it has the enzyme telomerase that makes telomeres. In theory a stem cell could live forever (and we also can live forever). But then why do we age and lose the numbers of available stem cells? According to the research “Stem cell function and maintenance—ends that matter: role of telomeres and telomerase” published by Stanford University School of Medicine in September 2013, our stem cells make less enzyme telomerase as we age. Consequently, stem cells do not live forever, and the older we are, the fewer the numbers of stem cells are available to repair injuries and replace the death ones.

As shown in FIG. 2, stem cells are activated by the Astragalus membranaceus, wherein the stem cells are regenerated to repair injured tissues via three mechanisms: (1) the rejuvenation of stem cells, (2) the proliferation of stem cells, and (3) the differentiation of stem cells. In the first mechanisms, Astragalus can rejuvenate stem cells to make them survive much longer. Many researches have shown that Astragalus has the capability to increase telomerase activities in vivo. Specifically, Astragaloside IV and cycloastragenol, two chemicals found in Astragalus, are responsible for increasing the telomerase activities. Consequently, more stem cells would be available to heal the repeated injured lung tissue via the regeneration instead of the scar formation. In spite of the high frequently for exposing to a potential harmful environment, the turnover rate of epithelial cells in adult lung is much lower than that of epithelial cells in our skin or intestine, so if bronchial epithelium are repeatedly injured, they cannot be quickly regenerated; therefore, instead of healing, the bronchial epithelium tend to generate sub-epithelial fibrosis, smooth muscle hypertrophy, and goblet cell hyperplasia. When we are still young and there are still abundant stem cells in the lung epithelium, the lining of the airways are regenerated. But when we are getting older and the numbers of stem cells diminish, the only way they can repair the repeated injury from tobacco smoking, air pollutants, and chemical fume is by formation of thickening and narrowing airways just like those of scar tissue. When we increase the telomerase activity of the stem cells, we can delay the progression of COPD.

It is worth mentioning that many researches have shown that Astragalus is safe for a long term oral consumption. One may argue that if you increase the telomerase activity, you may increase the risk of cancer because cancerous cells also have the enzyme telomerase. According to a research published in the journal Aging Cell on August 2011, taking Astragalus will not increase the risk of cancer because it increases the enzyme telomerase of only the stem cells but not the other normal mature cells.

The second mechanism that Astragalus activates stem cells is by promoting stem cells proliferation. Making more stem cells is not necessarily making more needed mature cells. The stem cells can just lying around without being differentiated into mature cells to replace the death ones. We need to encourage these primitive stem cells to differentiate into mature lung cells by the process known as proliferation. And Astragalus is known to promote stem cells proliferation. For example, Astragalus was known to induce bone marrow stem cells into osteoblasts according to the research “Effect of astragalus polysaccharides on the proliferation and ultrastructure of dog bone marrow stem cells induced into osteoblasts in vitro”.

The third mechanism that Astragalus activates stem cells is by promoting stem cells differentiation, of which stem cells from one type of cells to differentiate and become the other types of cells. For example, Astragalus can induce the differentiation of bone marrow stem cells to become nerve cells according to the research “Effect of Astragalus mongholicus on inducing differentiations of rat bone marrow-derived mesenchymal stem cells into neurocyte-like cells”. This finding is very important because a human lung is a complex architecture with diversity of cell types and niches. For example, an adult human lung has 23 airway generations from trachea to alveolar ducts, which terminate in millions of alveoli. Each airway region has distinct structure and function, with different tissue and cell types. Larger airways are lined with ciliated columnar epithelium, goblet cells, basal cells, and neuroendocrine cells while the more distal bronchioles are lined with fewer ciliated cells and Clara cells. The alveoli are lined only by type I and II pneumocytes. The process of differentiation is very important to help repairing the injured tissue in the complex human lung.

It is worth mentioning that Astragalus provides three miraculous properties. First of all, it is a potent antioxidant that helps to reduce airway injury. Secondly, it decreases the activity of the goblet cells so as to reduce mucus production so as to alleviate the symptoms of cough. Finally, it is an activator for the stem cells.

According to the preferred embodiment of the present invention, in order to alleviate the symptoms of COPD and delay the progression of this disease, both Curcumin and Astragalus should be used together for the following reasons. To begin with, both Curcumin and Astragalus are potent antioxidants. The use of the two together would result in better synergistic effect in reducing airway injury. Furthermore, both help to alleviate the symptoms of COPD and delay the progression of this disease by different mechanisms. In a short term, while Astragalus decreases the production of mucus by goblet cells, Curcumin decreases the secretion of the enzyme elastase. One helps to alleviate the symptoms of COPD in chronic bronchitis, the other in emphysema. Finally, both Curcumin and Astragalus have long term benefits in the treatment of COPD. Curcumin helps to reverse the epigenetic modification caused by the chronic exposure to tobacco smoking, air pollution, and chemical fume, and Astragalus helps to activate stem cells to repair the injured lung tissues. Both should be used together to have beneficiary effect because according to a research published on March 2014 by the University of Vermont, College of Medicine, USA, normal lung cells and emphysematous lung cells recellularize differently. In this experiment, cadaveric human lungs from a normal non-smoking person and from a smoker with COPD (emphysema) were decellularized (extracting and getting rid of cells) but retained the characteristic histological architecture and extracellular matrix components. Stem cells were inoculated to grow on these two molded matrixes. It was found that the stem cells growing on the decellularized normal lung can survive up to one month. In contrast, stem cells inoculated into decellularized emphysematous lungs did not survive beyond one week. The chronic exposure to tobacco smoking has methylated or acetylated the histones and epigenetically turned on or off certain segments of the DNA. Typical stem cells therapy without reversing the epigenetic modification would not have good results.

Accordingly, the therapeutic nutritional supplement of the present invention can be utilized for therapeutic drugs, medications, herbal supplements in a form of tablets, capsules, and soft-gels, a beverage including coffee, tea, energy drinks, juice, water, and so on, and foods including in gummies or any gelatin-based products, such as cookies, cakes, and ice-cream. It is intended to alleviate the symptoms of chronic obstructive pulmonary disease (COPD) and delay the progression of this disease.

According to the preferred embodiment of the present invention, Astragalus and Curcumin can be used together to achieve the better efficacy of the therapeutic nutritional supplement. The therapeutic nutritional supplement can also be used with prescribed or over-the-counter medications including inhaled or oral bronchodilators such as β₂ agonists and anticholinergic, inhaled or oral corticosteroids, antibiotics, methylxanthines (theophylline), mucolytic, supplemental oxygen, and cough medicines. The therapeutic nutritional supplement can also be used with other herbal supplements that have known for having effective treatments for asthma, COPD, coughing, allergy, allergic rhinitis, and shortness breathing, wherein such herbal supplements can be extracted from pine bark (Procyanidins), ginger root extract, mullein, ivy leaf extract, and cordyceps.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A therapeutic nutritional supplement to alleviate symptoms of chronic obstructive pulmonary disease (COPD) and delay the progression thereof, comprising:
a predetermined amount of Astragalus having a daily dosage range from 100 mg to 2000 mg; and
a predetermined amount of Curcumin having a daily dosage range from 10 mg to 300 mg.

2. The supplement, as recited in claim 1, wherein said Astragalus is an antioxidant having anti-inflammatory properties, is an inhibitor for goblet cells, and is an activator for stem cells.

3. The supplement, as recited in claim 1, wherein said Curcumin is an antioxidant having anti-inflammatory properties, is an inhibitor for a production of enzyme elastase, and is able to reverse an epigenetic modification process.

4. The supplement, as recited in claim 1, wherein said daily dosage of said Astragalus is 500 mg, and said daily dosage of said Curcumin is 50 mg.

5. The supplement, as recited in claim 1, further comprising a flavanols Procyanidins serving as an elastase-inhibiting agent being used together with said Curcumin to perform a synergistic effect with said Curcumin.

6. The supplement, as recited in claim 5, wherein said Procyanidins has a daily dosage range from 10 mg to 300 mg.

7. The supplement, as recited in claim 6, wherein said daily dosage of said Procyanidins is 50 mg.

8. The supplement, as recited in claim 5, wherein said Procyanidins is extracted from a group consisted of apples, maritime pine bark, cinnamon, aronia fruit, cocoa beans, grape seed, grape skin, bilberry, cranberry, black currant, green tea, black tea, and wine barrel oaks.

9. The supplement, as recited in claim 1, further comprising red reishi for providing an anti-allergic effect.

10. The supplement, as recited in claim 9, wherein said daily dosage form said red reishi is 275 mg.

11. The supplement, as recited in claim 1, which is used as therapeutic drugs, medications, and herbal supplements in form of tablets, capsules, liquid, syrups, and soft-gels.

12. The supplement, as recited in claim 1, which is mixed with beverages and foods.

13. The supplement, as recited in claim 1, which is used with prescribed or over-the-counter medications including inhaled or oral bronchodilators, such as β₂ agonists and anticholinergic, inhaled or oral corticosteroids, antibiotics, methylxanthines (theophylline), mucolytic, supplemental oxygen, and cough medicines.

14. The supplement, as recited in claim 1, which is used with other herbal supplements that have known for having effective treatments for asthma, COPD, coughing, allergy, allergic rhinitis, and shortness breathing.

15. The supplement, as recited in claim 1, which is used with exercise regimen and the rehabilitation of pulmonary.

16. A method for alleviating symptoms of chronic obstructive pulmonary disease (COPD) and delaying the progression thereof, comprising the steps of:
(a) administering a predetermined amount of Astragalus having a daily dosage range from 100 mg to 2000 mg; and
(b) administering a predetermined amount of Curcumin having a daily dosage range from 10 mg to 300 mg.

17. The method, as recited in claim 16, which is used as therapeutic drugs, medications, and herbal supplements in form of tablets, capsules, liquid, syrups, and soft-gels.

18. The method, as recited in claim 16, which is mixed with beverages and foods.

19. The method, as recited in claim 16, wherein said daily dosage of said Astragalus is 500 mg, and said daily dosage of said Curcumin is 50 mg.

20. The method, as recited in claim 16, further comprising a step of administering a flavanols Procyanidins serving as an elastase-inhibiting agent being used together with said Curcumin to perform a synergistic effect with said Curcumin.

21. The method, as recited in claim 20, wherein said Procyanidins has a daily dosage range from 10 mg to 300 mg.

22. The method, as recited in claim 16, further comprising a step of administering red reishi for providing an anti-allergic effect.

23. The method, as recited in claim 22, wherein said daily dosage form said red reishi is 275 mg.
